(19) United States
(12) Patent Application Publication
(10) Pub. No.: US 2005/0014688 A1
(43) Pub. Date: Jan. 20, 2005

(54) Inducer of apoptosis

(76) Inventor: Kevin Gaston, Bristol (GB)

Correspondence Address: DAVIS WRIGHT TREMAINE, LLP, 2600 CENTURY SQUARE, 1501 FOURTH AVENUE, SEATTLE, WA, 98101-1688, US

(21) Appl. No.: 10/484,644

(22) PCT Filed: Jul. 23, 2002
(86) PCT No.: PCT/GB02/03360

(30) Foreign Application Priority Data
Jul. 23, 2001 (GB) 0117905.0

(51) Int. Cl. A61K 38/17; A61K 48/00

(52) U.S. Cl. 514/12; 514/44

(57) ABSTRACT

This invention relates to the use of a mutant E2 polypeptide to induce apoptosis (a form of programmed cell death) in cells. The mutant E2 derived polypeptide is p53 binding deficient. Preferably it is unable to bind p53; or able to bind p53 but unable to induce p53-dependent apoptosis.

### FIELD OF THE INVENTION

This invention relates to a method of inducing apoptosis (a form of programmed cell death), particularly in cells that contain papillomavirus DNA, using a mutated papillomavirus E2 protein; to methods of killing cells using such mutant proteins and to E2 derived polypeptides.

### BACKGROUND

Papillomaviruses (PV) are DNA viruses that have a double stranded circular genome containing several open reading frames (ORFs) which encode products including the E6, E7, and E2 proteins (Meyers, G., et al. (1995) Human papillomaviruses, 1995 compendium. Los Alamos National Laboratory, Los Alamos, N. Mex., USA). These viruses infect epithelial cells and induce the formation of hyperproliferative lesions in which the viral DNA is usually present as an episome “i.e. autonomous self replicating DNA” (Lorincz A. T. et al. (1992) Obstet. GynecoL 79, 328-337). At least 95 different types of human PV (HPV) have been identified, many of which infect the genital tract and produce genital warts (Van Ranst M., et al (1992) J. Gen. Virol. 73, 2653-2660). Other HPV types are associated with cancer. For instance, HPV DNA can be detected in virtually all cervical cancers (99.7%) and these viruses are generally acknowledged to be the causative agent of this disease (Walboomers J. M., et al. (1999) J. Pathol. 189, 12-19). HPVs are also thought to be involved in a variety of other diseases including: cancer of the vulva, oral cancer, skin cancer, and cancer of the esophagus (Basta A., et al. (1999) Eur. J. Gynaecol Oncol. 20, 111-114: Miller C. S., & Johnstone B. M. (2001) Oral Surg. Oral Med Oral Pathol. Oral Radiol. Endod. 91, 622-635: Biliris K. A., et al. (2000) Cancer Lett. 161, 83-88: Layergne D., & de Villiers E. M. (1999) Int. J. Cancer 80, 681-684).

In contrast to the episomal HPV DNA present in HPV-infected cells, the HPV DNA present in HPV-transformed cancer cells is often integrated into the host genome (Dürst M., et al. (1985) J. Gen. Virol. 66, 1515-1522: Kalantari M., et al. (2001) Diagn. Mol. Pathol. 10, 46-54). However, cervical cancer cells continue to express the HPV E6 and E7 ORFs and the products of these oncogenes act to increase cell proliferation and promote cell immortalization (Hawley-Nelson, P., et al. (1989) EMBO J. 8, 3905-3910: Scheffner, M., et al. (1990) Cell 63, 1129-1136: Dyson, N., et al. (1989) Science 243, 934-936), essential initial steps in tumourigenesis.

The papillomavirus E2 ORF encodes a sequence-specific DNA binding protein that regulates viral gene expression and which is also required for efficient viral DNA replication (Bouvard V., et al. (1994) EMBO J. 13, 5451-5459: Frattini M. G. & Laimins L. A. (1994) Proc. Natl. Acad. Sci. USA 91, 12398-12402: Berg M. & Stenlund A. (1997) J. Virol. 71, 3853-3863). The E2 protein regulates transcription of the E6 and E7 oncogenes and can thereby affect cell proliferation (Dowhanick J. J., et al. (1995) J. Virol. 69, 7791-7799: Sanchez-Perez A. M., et al. (1997) J. Gen. Virol. 78, 3009-3018: Desaintes C., et al. (1999) Oncogene 18, 4538-4545). The integration of HPV DNA into the host genome often disrupts the E2 ORF and/or blocks the correct expression of E2 (Dürst M., et al. (1985) J. Gen. Virol. 66, 1515-1522: Rose B. R., et al. (1997) Gynecol. Oncol. 66, 282-289). This is thought to lead to over-expression of E6 and E7, which in turn leads to tumourigenesis. Consistent with this view, over-expression of E2 proteins in cervical cancer cells can repress E6 and E7 expression, resulting in apoptotic cell death and growth suppression (Dowhanick J. J., et al. (1995) J. Virol. 69, 7791-7799: Francis D. A., et al. (2000) J. Virol. 74, 2679-2686: Nishimura A., et al. (2000) J. Virol. 74, 3752-3760). This finding gave rise to the proposal that E2 could be useful in the treatment of cervical cancer and other HPV-induced diseases. However, the E2 protein can also induce apoptosis in cells that do not contain HPV DNA (Webster K., et al. (2000) J. Biol. Chem. 275, 87-94).

p53 is a cellular tumour suppressor protein that is inactivated by mutation in around half of all human tumours (reviewed by Cox L. S. & Lane D. P. (1995) Bioessays 17, 501-508). In response to a number of stimuli including ionising radiation, cell stress, or viral infection, the p53 protein can mediate either cell cycle arrest or apoptosis. Cells from HPV-induced tumours usually contain wild-type p53 and can respond to signals that induce p53 activity (Butz K., et al. (1995) Oncogene 10, 927-936: Webster K., et al. (2000) J. Biol. Chem. 275, 87-94). The E2 protein from at least one HPV type binds to p53 (Massimi P., et al. (1999) Oncogene 18, 7748-7754) and this E2 protein can also induce p53-dependent apoptosis (Webster K., et al. (2000) J. Biol. Chem. 275, 87-94). Here we show that a mutant of the E2 protein that binds weakly to p53 is capable of inducing apoptosis in HPV-transformed cells but is incapable of inducing apoptosis in non-HPV transformed cells. This novel mutant of E2 could be useful in cancer therapy, in the treatment of precancerous lesions, and in the treatment of HPV infections. In particular the invention provides a treatment for HPV infection, particularly the pre-cancerous condition known as cervical intraepithelial neoplasia. Currently, many women diagnosed with this condition are not immediately treated but are regularly monitored for progessing infection. Early treatment using the methods of this invention would alleviate much stress as well as reducing the possibility of deterioration to a more serious form of the pre-cancerous condition which has a higher risk of developing into cancer of the cervix. In contrast to such active treatments the methods and compositions can be employed prophylactically against cervical cancer.

WO00/02693 (The University of Bristol) discloses methods and compositions for inducing cell death in HPV-transformed and non-HPV-transformed cells using E2 polypeptides. There is no disclosure of methods or mutations that could target E2-induced apoptosis to HPV-transformed cells or HPV-infected cells.

WO98/01148 (Harvard) discloses methods and compositions for interfering with the proliferation of cells infected with and/or transformed by PV. There is no disclosure of the p53 status of the cells, the induction of apoptosis in the treated cells, or the effects of E2 on HPV-negative cells.

WO94/04686 (Biogen) describes a method for the delivery of proteins, including HPV E2 polypeptides, to cells based on the HIV TAT protein. There is no disclosure of the p53 status of the cells, the induction of apoptosis in the treated cells, or the effects of E2 on HPV-negative cells.

WO92/12728 (Biogen) discloses non-functional E2-derived polypeptides, specifically E2 trans-activation repressors, which form heterodimers with normal E2 and block its function in HPV-infected cells. There is no disclosure of the p53 status of the cells, the induction of apoptosis in the treated cells, or the effects of E2 on HPV-negative cells.

### DISCLOSURE OF THE INVENTION

According to one aspect of the invention there is provided a method of killing cells that contain HPV DNA, comprising contacting the cells with a p53 binding-defective PV E2-derived polypeptide. Such a polypeptide binds to p53 with less affinity (if at all) than a wild-type or native HPV 16 E2 protein. The cells may be PV-transformed or PV-infected. The invention applies to all HPV types.

According to another aspect of the invention there is provided a method of inducing apoptosis in PV-transformed cells and/or PV-infected cells comprising contacting the cells with a p53-binding-defective PV E2-derived polypeptide.

According to another aspect of the invention there is provided a method of killing PV-transformed cells and/or PV-infected cells comprising contacting the cells with a DNA sequence encoding a p53-binding-defective PV E2-derived polypeptide.

According to another aspect of the invention there is provided a method of killing PV-transformed cells and PV-infected cells comprising contacting the cells with a p53 binding-defective PV E2-derived protein that is unable to bind p53 or a functional portion thereof.

According to a further aspect of the invention there is provided a method of killing PV-transformed cells and PV-infected cells comprising contacting the cells with a nucleotide sequence encoding a p53-binding defective PV E2-derived polypeptide fused to VP22 or a derivative thereof. VP22 is a Herpes Simplex Virus-1 protein that can be used to efficiently deliver other polypeptides to mammalian cells (Elliot, G and O'Hare, P. (1997) Cell, 88: 223-233 and WO00/53722). It is advantageous because it should allow the delivery of the E2-derived protein to a large number of cells compared to conventional gene therapy techniques. Suitable derivatives of or alternatives to VP22 having a similar transport function may be determined by the skilled worker. Other non-viral methods of delivering E2 derived polypeptides include penetratin or liposomes. Viral methods of delivering E2 derived polypeptides in adenovirus, adeno-associated virus, retrovirus, and pox virus. Preferably, the nucleotide sequence encodes a p53-binding defective PV E2-derived polypeptide fused to VP22. More preferably, the p53-binding defective E2 derived polypeptide is delivered as a histidine-tagged VP22 fusion protein. Most preferably the nucleotide sequence encodes a PV E2-derived polypeptide, that is unable to bind p53, fused to histidine-tagged VP22.

According to another aspect of the invention there is provided the use of a p53-binding defective PV E2-derived polypeptide in a pharmaceutical composition for the active or prophylatic treatment or amelioration of cervical cancer. The pharmaceutical composition would comprise suitable diluent or carrier. It is preferred that the composition is in the form of a cream or spray which can be applied directly to an infected area especially the cervical area in a female human subject.

According to another aspect of the invention there is provided the use of a p53 binding defective E2-derived polypeptide in a pharmaceutical composition for the active or prophylactic treatment or amelioration of genital warts.

According to another aspect of the invention is provided the use of a DNA sequence encoding a p53-binding defective E2-derived polypeptide in a pharmaceutical composition for the active or prophylactic treatment or amelioration of cervical cancer.

According to another aspect of the invention there is provided the use of a DNA sequence encoding a p53-binding defective E2-derived polypeptide in a pharmaceutical composition for the active or prophylactic treatment or amelioration of genital warts.

According to another aspect of the invention there is provided the use of a p53 binding defective E2-derived polypeptide in a pharmaceutical composition for the treatment or amelioration of precancerous lesions.

According to another aspect of the invention is provided the use of a DNA sequence encoding a p53-binding defective E2-derived polypeptide in a pharmaceutical composition for the treatment or amelioration of precancerous lesions.

Preferably a substantial portion, for example greater than 50%, preferably more than 80%, most preferably more than 90%, of the cells are killed in a method of inducing apoptosis or killing cells according to the invention.

Preferably the cells in which apoptosis is induced are tumourigenic. Where the cells are cancerous, the cancer may be selected from cervical cancer, cancer of the vulva, oral cancer, cancer of the oesophagus.

The HPV DNA may be integrated into the cell genome or may be present in the cell as episomes. Alternatively, the HPV DNA may be present in the cell as both episomes and integrated DNA.

The cells may be mammalian. The cells may be cervical, vulval or epidermal. Preferably, the cells are human cells. More preferably the cells are in a mammalian subject, most preferably a human subject.

In methods and uses in accordance with the invention, the p53-binding defective E2-derived polypeptide may be able to bind p53 but unable to induce p53-dependent apoptosis.

According to another aspect of the invention there is provided a p53-binding defective E2-derived polypeptide for use in methods and uses of the invention in which the polypeptide is mutated at at least one of positions Asp338, E340 (Glu 340), Trp341, and Asp344 of the native HPV 16 protein amino acid sequence.⁹ Preferably the p53-binding defective E2-derived polypeptide is unable to bind p53. Where the polypeptide is able to bind p53, it may be unable to induce p53-dependent apoptosis. The PV from which the p53-binding defective polypeptide is derived may be HPV type 16, 18, 33, 31, 6, 11, 2, 4, 1, or 7. Preferably, the PV is an animal, most preferably a mammalian PV, especially human PV. The polypeptide may have the same or similar length as a native E2 protein or may be substantially shorter or longer. More particularly, where the polypeptide is shorter than a native E2 protein the N terminal portion of the E2 protein may be truncated by 10 to 20 amino acids whereas in the C terminal portion the truncation may be 20 to 40 amino acids. Where the polypeptide is longer than a native E2 protein it may be 10 or more amino acids longer than at the N terminal end—for example because it includes a histidine tag or is attached to VP22.

Alternatively the native sequence may be altered at one or any other positions that are important for the E2-p53 interaction. Mutations and alterations may be in the form of insertions, deletions or substitutions. Amino acids in a native sequence may be modified to enhance useful properties such as inhibition of p53 binding, stability, immunogenicity, expression. Polypeptides in accordance with the invention may be produced by recombinant organisms or chemically synthesized.

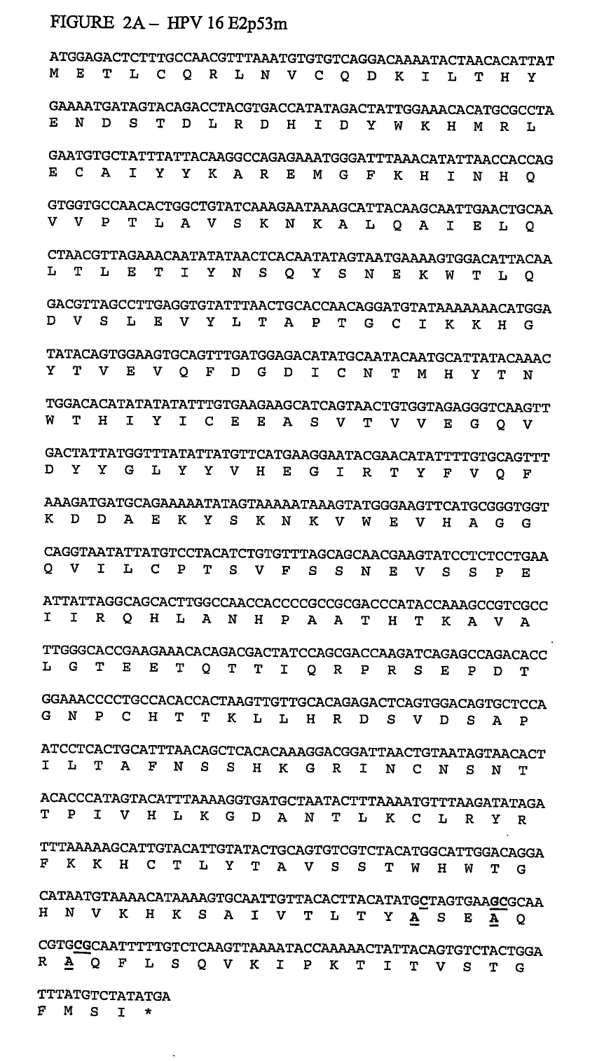

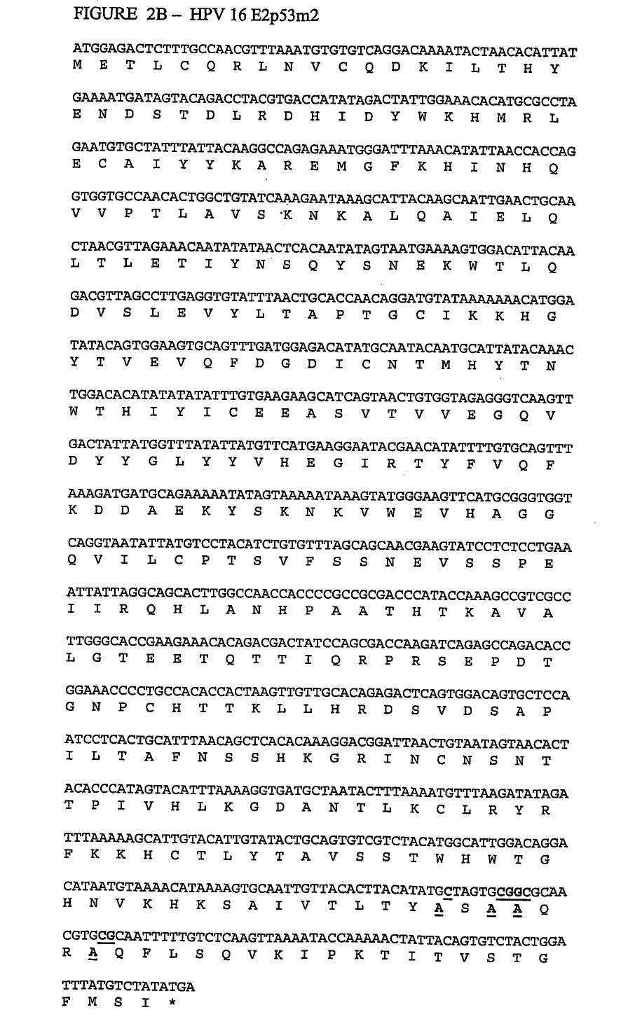

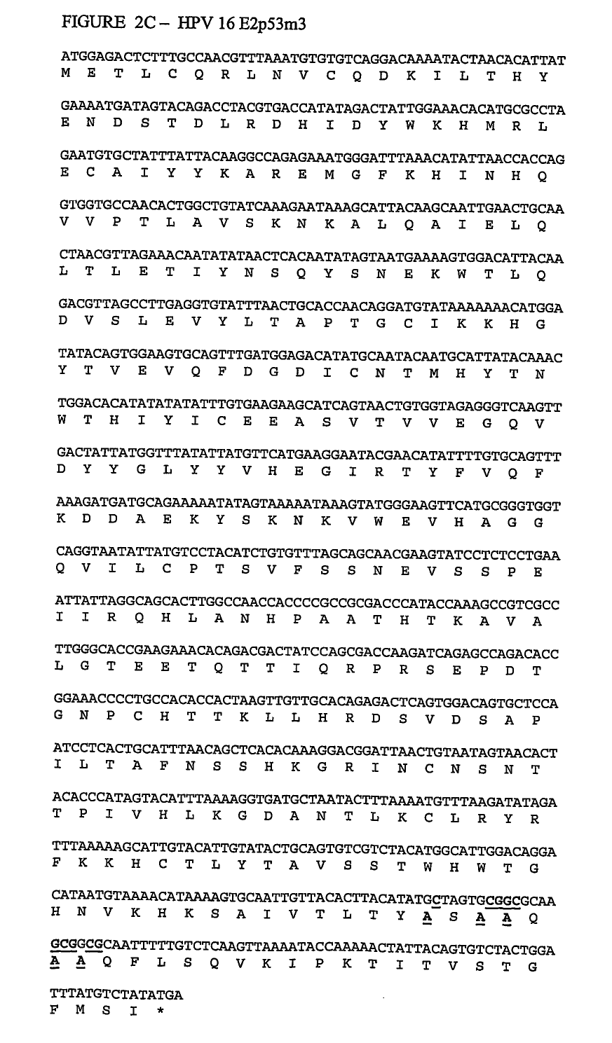

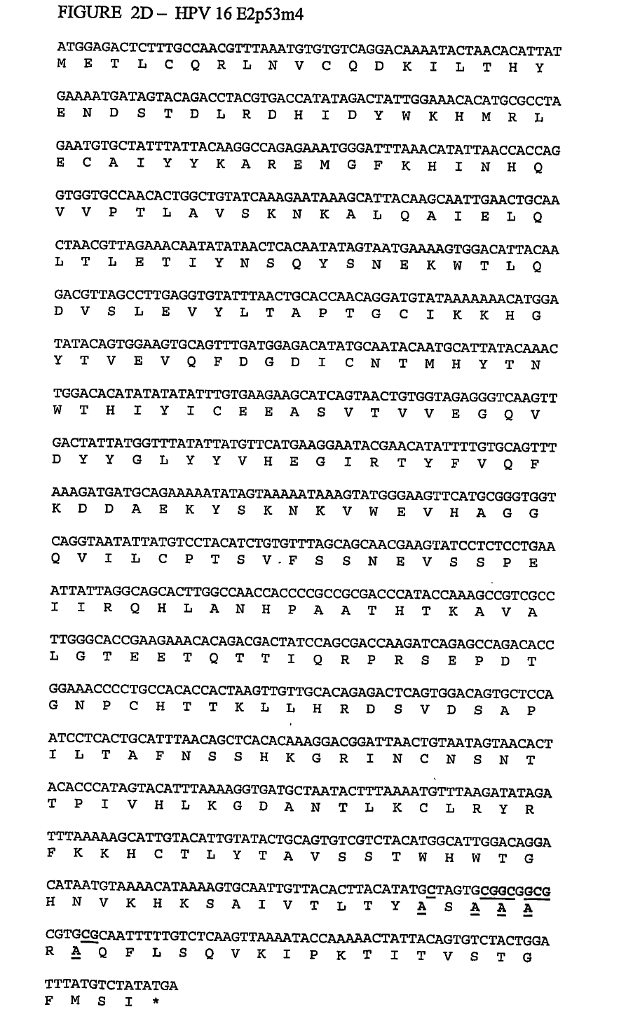

A preferred polypeptide in accordance with the invention has the amino acid sequence shown in FIG. 2A, 2B, 2C, or 2D.

The invention also provides nucleotide sequences encoding p53-binding defective E2-derived polypeptides of the invention. A preferred nucleotide has the nucleotide sequence shown in FIG. 2A, 2B, 2C, or 2D.

The nucleotide may encode an HPV 16 E2 derived polypeptide mutated at positions Asp338, Glu 340, Trp341, and Asp344 of the native sequence or any other positions that are important for the E2-p53 interaction.

The p53 binding-defective PV E2-derived polypeptide may be delivered as a VP22 fusion protein. Other delivery methods such as gene therapy and antibody delivery are contemplated. For example, adenovirus, adeno-associated virus, or retroviruses may be used. The skilled worker will be able to select suitable methods.

Definitions

In this specification, the following expressions are used with the following non-limiting meanings given by way of explanation:
1. “PV positive” a cell that has been infected or transformed by a PV. “PV negative” has an opposite meaning.
2. “Protein” and “polypeptide” are used interchangeably although protein may be considered to be a naturally occurring polypeptide.
3. “HPV DNA” means a DNA from an HPV comprising a complete open reading frame (ORF) or a significant amount, such as about 100 bp, of non-coding sequence from a naturally occurring HPV genome.
4. “Apoptosis” is a form of cell death chiefly though not exclusively characterised by plasma membrane blebbing, chromatin condensation, and the formation of apoptotic bodies (cell bodies with sub-GO DNA content).
5. “PV infected” cells are non-malignant (non tumourigenic) cells containing HPV DNA.
6. “PV transformed” cells are malignant cells that contain HPV DNA.

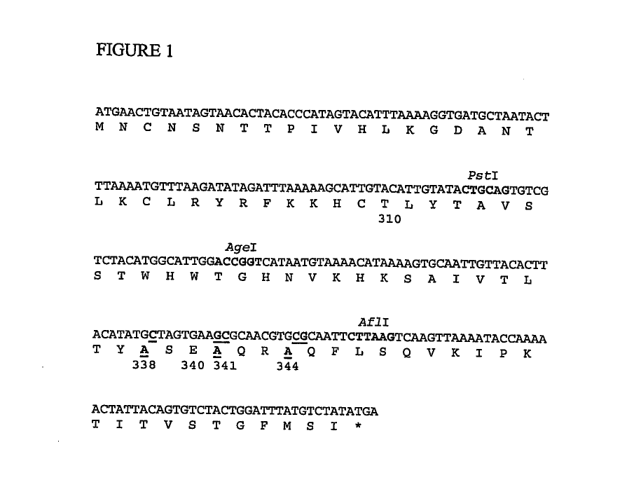

A method of inducing cell death and products in accordance with the invention will now be described, by way of example only, with reference to the accompanying drawings FIGS. 1 to 8 in which:

FIG. 1 shows the DNA for and amino acid sequence of a p53 binding-defective E2-derived polypeptide HPV 16 E2p53mCt. The mutated bases and amino acids are shown in bold and are underlined;

FIG. 2 shows the DNA for and amino acid sequence of HPV 16 E2p53m and other E2 derived polypeptides in accordance with the invention. The mutated bases and amino acids are shown in bold and are underlined;

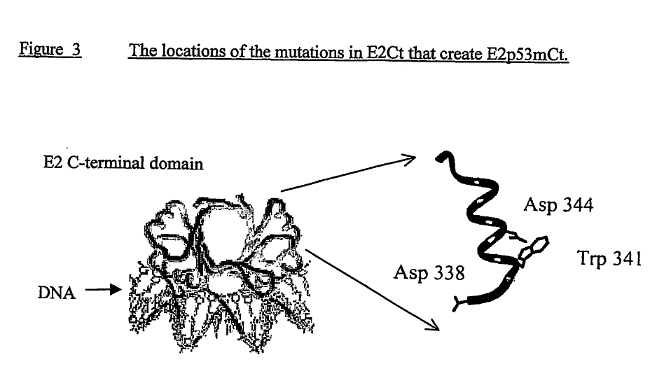

FIG. 3 shows the HPV 16 E2 DNA binding domain and the positions at which amino acids were mutated to create E2pS3m and E2p53mCt;

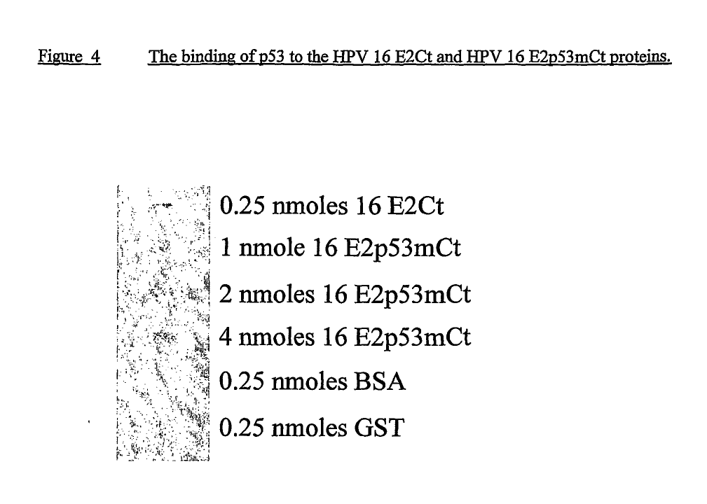

FIG. 4 shows the binding of labelled p53 to the E2Ct protein and the E2p53mCt protein;

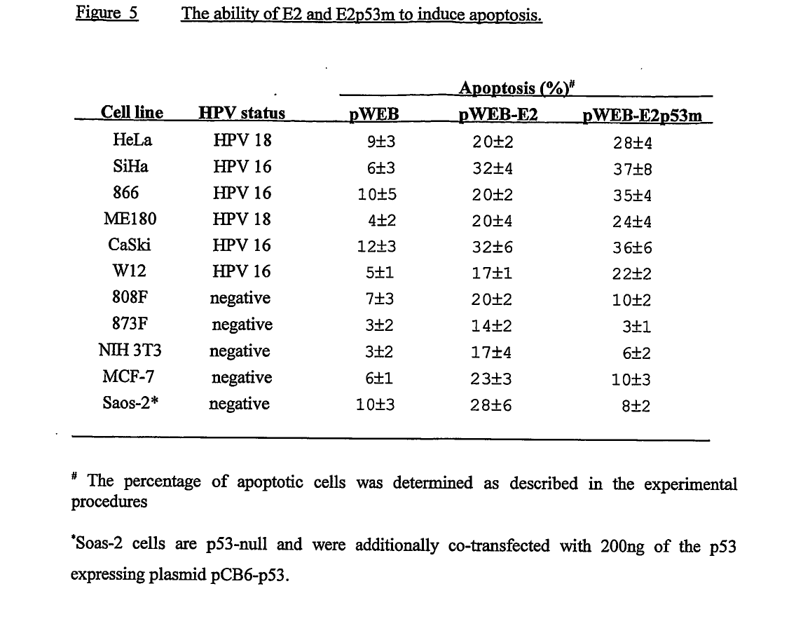

FIG. 5 shows the effects of the E2 and E2p53 proteins on a variety of HPV-transformed and non-HPV transformed cell lines;

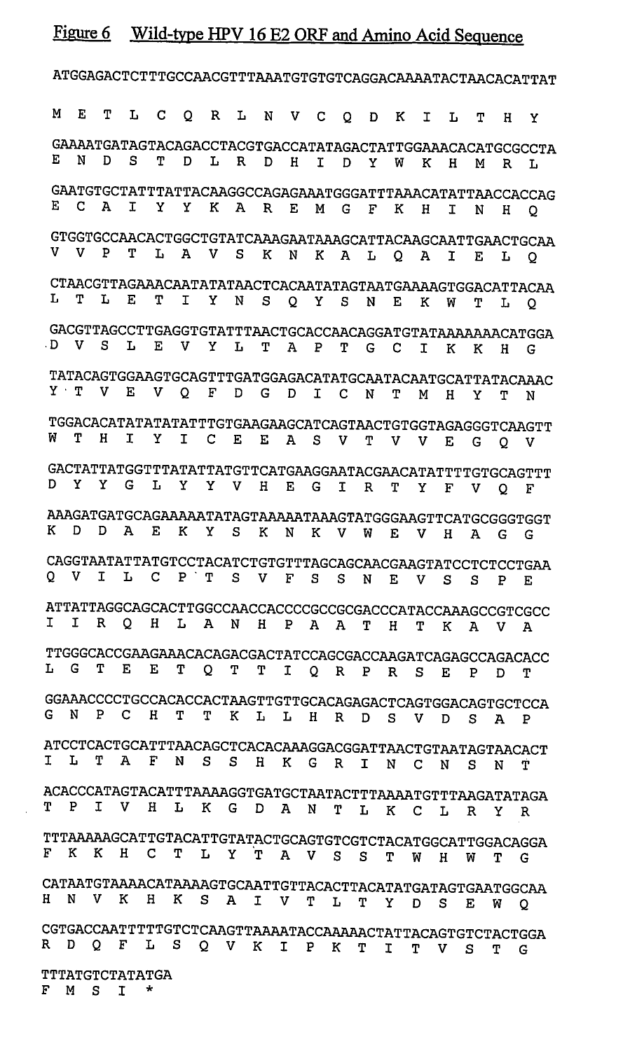

FIG. 6 shows the ORF and amino acid sequence of HPV16 E2;

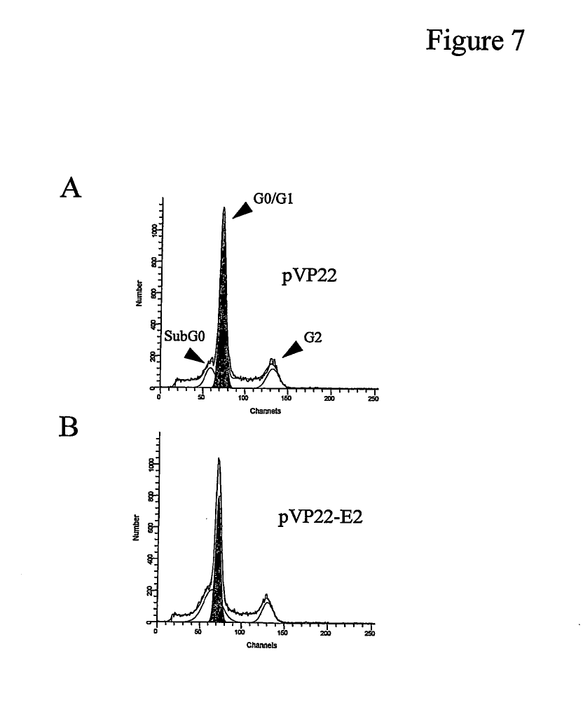

FIG. 7 shows the effects of VP22 and VP22-E2 fusion proteins on HPV-transformed cells; and

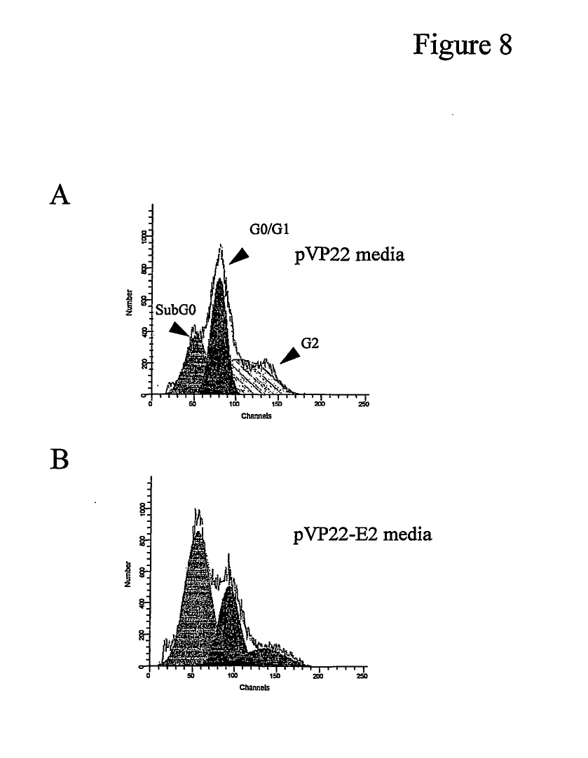

FIG. 8 shows the effects of a VP22-E2 fusion protein produced in E2-insensitive COS-7 cells on HPV-transformed cells.

### 1. EXPERIMENTAL PROCEDURES—GENERAL

a. Plasmids

The plasmids pWEB-E2 and pCMX-GFP3 express the HPV 16 E2 protein and the green fluorescent protein, respectively (Webster K., et al. (2000) J. Biol. Chem. 275, 87-94). The p53 expression vector pCB6-p53 was supplied by Dr K. Vousden (NCI-FRDC, USA). The plasmid pBluescript-p53 contains the wild-type p53 coding sequence downstream of the T7 promoter. pBluescript-p53 was created by cloning a BamHI fragment carrying the p53 cDNA from pC53-SN3 (supplied by Dr B. Vogelstein (Johns Hopkins Oncology Center, USA)) into the unique BamHI site in pBluescript II (Stratagene). The plasmid pKK-E2Ct expresses the DNA binding domain of the HPV 16 E2 protein (amino acids 280 to 365) in bacterial cells (Webster K., et al. (2000) J. Biol. Chem. 275, 87-94). The plasmid pKK-E2p53mCt was created by replacing the E2 sequences between the unique PstI and HindIII sites in pKK-E2Ct with three double stranded synthetic oligonucleotides with complementary ends. The synthetic oligonucleotides introduced three amino acid changes into the E2 sequence: Asp338 to alanine, Trp341 to alanine, and Asp344 to alanine (FIG. 1). Further changes that are contemplated include Glu340 to alanine, Gln342 to alanine, Gln345 to alanine, and Arg343 to alanine. The plasmid pWEB-E2p53m was created by replacing the PstI-EcORI region of pWEB-E2, encoding the C-terminal region of E2, with the corresponding region of pKK-E2p53m encoding the mutated C-terminal region of E2 (FIG. 2). The plasmid pVP22 (Invitrogen) encodes the Herpes Simplex Virus-1 VP22 protein. DNA sequences encoding the E2 and E2p53m proteins were cloned into pVP22 in frame with the VP22 coding sequence. All constructs were sequenced to check for the presence of any unwanted mutations.

b. Protein Purification

The E2Ct and E2p53mCt proteins were expressed in bacteria and purified exactly as described previously (Webster K., et al. (2000) J. Biol. Chem. 275, 87-94).

c. Protein-Protein Interactions

In vitro transcription and translation of p53 cloned in pBluescript-p53 was carried out using a TNT kit (Promega) according to the manufacturer's instructions. The HPV 16 E2Ct and HPV 16 E2p53mCt proteins were immobilized on PVDF membranes by slot blotting. After staining with 0.1% w/v Ponceau S (Sigma) to conform immobilization the membranes were washed three times in Tris-buffered saline, 0.02% v/v Tween 20, 10% w/v dried skimmed milk powder (20 minute washes at 22° C.). The membranes were then incubated with 25 μl of ³⁵S-labelled p53 in 10 ml of Tris-buffered saline, 0.02% v/v Tween 20, 10% w/v dried skimmed milk powder (90 minutes at 22° C.). The membranes were then dipped in methanol before being left to dry on Whatman paper (22° C. for 15 minutes). Bound labelled p53 was visualized using a PhosphorImager.

The affinity of E2 derivatives in accordance with the invention for p53 may be quantitatively assesed by surface plasma resonance. More particularly, a GST-p53 fusion protein is captured on a BIACORE Sensor Chip CM5 flow cell surface that has previously been coated with GST antibodies using an Amine Coupling Kit (BIACORE). Purified E2 protein and E2 mutants can then be applied to the surface and their binding assayed using surface plasmon resonance (Buckle M., et al (1996) Proc. Nat. Acad. Sci. (USA) 93, 889-894).

d. Cell Culture and Transfections

HeLa (HPV18 transformed cervical carcinoma cells), SiHa, CaSki, MEI80, NIH3T3, Saos-2, and MCF-7 cells were maintained in Dulbecco's Modified Eagle's Medium (DMEM) supplemented with 10% foetal bovine serum (FBS) and penicillin (10⁵ units L⁻¹) and streptomycin (100 mg L⁻¹). 866 cells were maintained in DMEM supplemented with 5% FBS insulin (5 μg μl⁻¹), hydrocortisone (0.01 μg ml⁻¹), penicillin (10⁵ units L⁻¹) and streptomycin (100 mg L⁻¹). 808F and 873F cells were maintained in DMEM supplemented with 5% FBS, insulin (5 μg ml⁻¹), epidermal growth factor (EGF) (0.01 μg ml⁻¹) cholera toxin (0.01 μg ml⁻¹), hydrocortisone (0.4 μg ml⁻¹), penicillin (10⁵ units L⁻¹) and streptomycin (100 mg L⁻¹). W12 cells were maintained in DMEM, 10% FBS, cholera toxin (0.01 nM), hydrocortisone (0.4 μg ml⁻¹), EGF (0.01 μg ml⁻¹), penicillin (10⁵ units L⁻¹) and streptomycin (100 mg L⁻¹). W12 cells required 3T3 cell feeder support. All cells were maintained in a humidified atmosphere at 37° C. and 5% CO₂.

HeLa cells and ME180 cells are HPV18 transformed cervical carcinoma cells; SiHa and CaSki cells are HPV16 transformed cervical carcinoma cells, Nih3T3, Sao52, and ncF-7 cells are non-HPV transformed cell lines.

For microscopy: twenty four hours prior to transfection the cells were seeded at a density of 3×10⁵ cells per well onto coverslips in six-well plates and incubated overnight. The liposome based transfection reagents, Tfx-20 for 866, Saos-2, and CaSki cells, and Tfx-50 for NIH3T3 and SiHa (Promega) were used at a ratio of 3:1 liposome: DNA in 1 ml of serum-free media per transfection. The remaining cell lines were transfected using Fugene 6 (Roche). Following transfection and incubation for 30 hours, the cells were washed with phosphate-buffered saline (PBS) and fixed using 4% paraformaldehyde for 30 minutes in the dark. After a further wash with PBS, the cells were stained with bisbenzimide (Hoechst no. 33258, Sigma) for 30 minutes in the dark. Finally, the cells were washed with PBS and inverted onto microscope slides with 15 μl of MOWIOL.

For flow cytometry: HeLa cells (2.3×10⁶) were seeded in 75 cm³ flasks and incubated for twenty four hours prior to transient transfection using Fugene 6 (Roche).

e. Microscopy and Imaging

Fluorescence microscopy was carried out using a Leica DM IRBE inverted epi-fluorescent microscope with FITC and DAPI filter sets and a 20×air objective (Leica).

f. Flow Cytometry

Twenty four hours post-transfection HeLa cells were trypsinized and harvested by centrifugation. Floating (dead) cells were harvested from the media. The trypsinized and floating cells were pooled and then washed twice with PBS before being resuspended in 1 ml of ice-cold methanol and incubated at −20° C. for 5 minutes. After centrifugation (10 minutes/2500 rpm in a bench-top centrifuge), the cells were resuspended in 3 ml of PBS containing 50 μg ml⁻¹ propidium iodide (Sigma) and incubated at 4° C. for 30 minutes. After re-centrifugation the cells were resuspended in 500 μl of PBS and kept in the dark until analysis by flow cytometry (Becton Dickinson FACScalibur).

2. Results.

Blocking the Interaction of E2 and p53

p53 binds to the C-terminal DNA binding domain of the E2 protein (Massimi P., et al. (1999) Oncogene 18, 7748-7754). The structure of this domain of E2 bound to DNA has been determined by X-ray crystallography (Hegde R. S. & Androphy E. J. (1998) J. Mol. Biol. 284, 1479-1489). A molecular model of the E2-p53 interaction was made using the co-crystal structure of p53 and the p53-binding protein 53BP2 as a guide (Gorina S, & Pavletich NP. (1996) Science 274, 1001-1005). The modelling identified amino acids in the E2 protein (including Asp338, Trp341, and Asp344) that can be superimposed on amino acids present in 53BP2 and are important in the p53-53BP2 interaction (FIG. 3). Asp338, Trp341, and Asp344 in E2Ct were mutated to alanine using site-directed mutagenesis. The replacement of an amino acid residue with alanine is considered to be a neutral solution. The p53 binding ability of an E2 derivative in accordance with the invention may be further reduced by the introduction of an oppositely-charged residue which actively repels the p53 molecule. The ability of the mutated E2p53mCt protein to bind p53 in vitro was tested as described in section lc (FIG. 4). Although labelled p53 binds to the wild-type E2Ct protein, labelled p53 binds weakly, if at all, to the E2p53mCt protein.

E2p53m Induces Apoptosis in HPV-Transformed Cells but not in non-HPV -Transformed Cells.

The ability of E2-p53m to induce apoptosis was investigated by introducing the Asp338, Trp341, and Asp344 mutations into the full length E2 protein and transiently transfecting the construct into a variety of HPV-transformed and non-HPV transformed cell lines growing on coverslips (FIG. 5) (as described in Webster et al 2000 infra). Thirty hours post-transfection the cells were fixed and their DNA was stained with bisbenzimide (Hoechst stain). The transfected cells were identified on the basis of their green fluorescence upon excitation through a fluorescein isothiocyanate filter set. These cells were examined for chromatin condensation and membrane blebbing, two characteristic features of apoptosis, using Hoechst stain and GFP, respectively (Webster K., et al. (2000) J. Biol. Chem. 275, 87-94). One hundred transfected cells were counted on each coverslip and the experiment was repeated three times. All of the cell lines show a background level of apoptosis of between 3 and 12%; cells transfected with the empty pWEB vector (FIG. 5, column 3). The wild-type E2 protein induces a significant increase in the level of apoptosis in all of the cell lines tested (FIG. 5, column 4). However, the E2p53m protein only induces a significant increase in the level of apoptosis in the HPV-transformed cells (FIG. 5, column 5). Importantly, both E2 and E2p53m induce apoptosis in W12 cells, a cell line that contains episomal HPV DNA.

VP22 can be Used to Deliver E2 to Target Cells.

To determine whether the Herpes Simplex Virus-1 VP22 protein could be used to deliver E2 and E2 mutants to target cells, DNA sequences encoding the E2 protein were cloned in frame into a VP22 expression vector (Invitrogen). Microscopy underestimates the number of apoptotic cells since dead cells detach from the substrate. In contrast, flow cytometry examines all of the cells, including the floating dead cells, and thus gives a better estimate of the number of apoptotic cells. To examine the effects of the VP22-E2 expression plasmid on cell survival, we transiently transfected the plasmid into HeLa cells and twenty four hours post transfection examined the entire population of cells using flow cytometry (FIG. 7). HeLa cells transfected with the VP22 vector show around 10% apoptotic cells (FIG. 7A). In contrast, HeLa cells transfected with the VP22-E2 expression vector show around 35% apoptotic cells (FIG. 7B). These data show that VP22-E2 fusion proteins are capable of inducing apoptosis in these cells. To determine whether VP22-E2 can move from cell to cell and induce apoptosis in the recipient (bystander) cells, we transiently transfected COS-7 cells with the VP22-E2 expression vector described above. The HPV 16 E2 protein does not induce apoptosis in COS-7 cells (Webster K., et al. (2000) J. Biol. Chem. 275, 87-94). COS-7 cells transiently transfected with the VP22-E2 expression or with a plasmid that expresses VP22 alone were cultured for 30 hours. Media from the transfected COS-7 cells was then removed and added to cultures of HeLa cells. After 24 hours, the HeLa cell populations exposed to these conditioned media were examined by flow cytometry. Media from COS-7 cells expressing VP22 alone brings about a small increase in the percentage of apoptotic HeLa cells; from around 8-10% in the untreated population to around 15-20% in the treated cells (FIG. 8A). In contrast, media from COS-7 cells expressing VP22-E2 brings about a dramatic increase in the number of apoptotic cells; from around 8-10% in the untreated cells to greater than 60% in the treated cells (FIG. 8B). These data suggest that VP22-E2 fusion proteins produced in COS-7 cells can enter the media from where they are capable of entering and inducing apoptosis in HeLa cells. Thus cell-cell contact is not required for the movement of VP22-E2 into non-producing cells.

Taken together these data suggest that the E2-p53 interaction is necessary for E2-induced cell death in non-HPV transformed cells. Since the E2 protein can induce apoptosis in HPV-transformed cells by altering the expression of E6 and E7, mutations that block the interaction of E2 with p53 result in an E2 mutant that only induces apoptosis in HPV-transformed cells. These data also show that VP22 can be used to deliver E2 proteins to target cells and that VP22-E2 fusion proteins produced in one cell are able to kill bystander cells.

1. A method of killing cells that contain HPV DNA, comprising contacting the cells with a p53 binding-defective PV E2-derived polypeptide.

2. A method according to claim 1 in which the cells are PV-transformed or PV infected.

3. A method according to claim 1 or 2 in which the HPV DNA is integrated into the cell genome

4. A method according to claim 1 or 2 in which the HPV DNA is present in the cells as episomes.

5. A method according to claim 1 or 2 in which the HPV DNA is present in the cell as both episomes and genome integrated DNA.

6. A method of inducing apoptosis in PV-transformed cells and/or PV-infected cells comprising contacting the cells with a p53 binding-defective PV E2-derived polypeptide.

7. A method of killing PV-transformed cells and/or PV-infected cells comprising contacting the cells with a p53 binding-defective DNA sequence encoding a p53 binding-defective PV E2-derived polypeptide.

8. A method of killing PV-transformed cells and PV-infected cells comprising contacting the cells with a p53 binding-defective PV E2-derived polypeptide.

9. A method of killing PV-transformed cells and PV-infected cells comprising contacting the cells with a nucleotide sequence encoding a p53-binding defective PV E2-derived polypeptide.

10. A method according to claim 9 in which the nucleotide sequence encodes a p53-binding defective PV E2-derived polypeptide fused to VP22.

11. A method according to claim 10 in which the p53-binding defective E2 derived polypeptide is delivered as a histidine-tagged VP22 fusion protein.

12. A method according to claim 8 in which the nucleotide encodes a PV E2-derived polypeptide that is unable to bind p53 fused to histidine-tagged VP22.

13. A method according to any preceding claim in which more than 50% of the cells are killed.

14. A method according to claim 13 in which more than 80% of the cells are killed.

15. A method according to claim 14 in which more than 90% of the cells are killed.

16. A method according to any preceding claim in which the cells in which apoptosis is induced are tumourigenic.

17. A method according to claim 16 in which the cells are cancerous and in which the cancer is selected from cervical cancer, cancer of the vulva, oral cancer or cancer of the oesophagus.

18. A method according to any preceding claim in which the cells are mammalian.

19. A method according to claim 18 in which the cells are human cells.

20. A method according to claim 18 or 19 in which the cells are in a mammalian subject.

21. A method according to any preceding claims and in which the p53-binding defective E2 derived polypeptide is able to bind p53 but unable to induce p53 dependent apoptosis.

22. The use of a p53-binding defective PV E2-derived polypeptide in a pharmaceutical composition for the treatment or amelioration of cervical cancer.

23. The use of a p53 binding defective E2-derived polypeptide in a pharmaceutical composition for the treatment or amelioration of genital warts.

24. The use of a DNA sequence encoding a p53-binding defective E2-derived polypeptide in a pharmaceutical composition for the treatment or amelioration of cervical cancer.

25. The use of a DNA sequence encoding a p53-binding defective E2-derived polypeptide in a pharmaceutical composition for the treatment or amelioration of genital warts.

26. A p53-binding defective E2-derived polypeptide

27. A p53 binding-defective E2-derived polypeptide according to claim 25 in which the polypeptide is mutated at at least one of positions Asp338, Glu340, Trp341, Glu342, Arg343, Asp344 and Glu345 of a native HPV 16 protein amino acid sequence.

28. A p53-binding defective E2-derived polypeptide according to claim 25 or 26 which is unable to bind p53.

29. A p53-binding defective polypeptide which is derived from HPV type 16, 18, 33, 31, 6, 11, 2, 4, 1, or 7proteins.

30. A polypeptide according to claim 25, 26,27 or 28 in which the PV is an animal PV.

31. A polypeptide according to claim 29 in which the PV is a mammalian PV.

32. A polypeptide according to any one of claims 25 to 30 which has the same or similar length as a native E2 protein.

33. A polypeptide according to any one of claims 25 to 30 which is substantially shorter or longer than a native E2 protein.

34. A polypeptide having the amino acid sequence shown in FIG. 2.

35. A fusion protein comprising a p53 binding-defective PV E2-derived polypeptide and a VP22 fusion sequence.

36. A polynucleotide encoding a p53-binding defective E2-derived polypeptides according to any one of claim 25 to 33.

37. A polynucleotide having the nucleotide sequence of FIG. 2.

38. A nucleotide encoding an HPV 16 E2 derived polypeptide mutated at positions Asp338, Glu340, Trp341, Glu342, Arg343, and Asp344 and Glu345 of the native sequence or any other positions that are important for the E2-p53 interaction.
